(19) United States
(12) Patent Application Publication
(10) Pub. No.: US 2024/0293248 A1
(43) Pub. Date: Sep. 5, 2024

(54) FORMATION OF CUSHIONING ARTICLES BY TENSION ACTIVATION

(71) Applicant: 3M INNOVATIVE PROPERTIES COMPANY, St. Paul, MN (US)

(72) Inventors: Audrey A. Sherman, Woodbury, MN (US); Thomas R. Corrigan, St. Paul, MN (US); Guido Hitschmann, Nuess (DE); Michael W.T. Mansholt, Koln (DE); Nicholas K. Lee, Minneapolis, MN (US)

(21) Appl. No.: 18/571,371

(22) PCT Filed: Jun. 14, 2022
(86) PCT No.: PCT/IB2022/055517

§ 371 Date: Dec. 18, 2023

Related U.S. Application Data
(60) Provisional application No. 63/213,800, filed on Jun. 23, 2021.

(51) Int. Cl. A61F 5/30; B32B 3/26; B32B 5/02; B32B 5/18; B32B 5/24; B32B 5/32; B32B 7/02; B32B 7/12

(52) U.S. Cl. CPC A61F 5/30; B32B 3/266; B32B 5/02; B32B 5/022; B32B 5/18; B32B 5/245; B32B 5/32; B32B 7/02; B32B 7/12; B32B 2262/0261; B32B 2266/0278; B32B 2307/558; B32B 2307/724; B32B 2535/00

(57) ABSTRACT

Cushioning articles include a layer with a plurality of cuts arrayed in a pattern. The cuts are gaps and the gaps do not form apertures allowing one to see through the layer when the layer is in an unstressed state, but in a stressed state, at least some of the cuts become gaps that are perforations through the layer. In the stressed state the layer expands to form the cushioning article. The cushioning article may be a multi-layer article with another layer that is contacted to the stressed layer in the stressed state to hold the layer in a stressed state.

### SUMMARY

Disclosed herein are cushioning articles suitable for medical applications. In some embodiments the cushioning article comprises a sheet-like conformable layer that is expandable, comprising a length in an x direction, a width in a y direction and thickness in a z direction, where the layer comprises a plurality of cuts. The plurality of cuts is arrayed in a pattern, and the cuts are gaps, the gaps are formed without the removal of material from the layer and the gaps do not form apertures allowing one to see through the layer when the layer is in an unstressed state, but in a stressed state, when stress is applied in either the x or y direction, the article becomes an expanded article where at least some of the cuts become gaps that are perforations through the layer. In a stressed state the perforations are apertures through which one can view through the layer, and the layer expands in the z direction such that the article, when disposed on a human body, is able to provide increased cushioning to the human body by the absorption of energy and has increased breathability as measured by MVTR (Moisture Vapor Transmission Rate), compared to an article that is in an unstressed state.

Also disclosed herein are medical articles that are multi-layer articles comprising a first layer, and a second layer in contact with the first layer where the second layer is thicker than the first layer. The second layer comprises a plurality of apertures, where the apertures are prepared by forming an array of cuts in the second layer without removing material from the second layer and applying stress to the second layer causing the cuts to form apertures. The first layer holds the second layer in a stressed state such that the article, when disposed on a human body, is able to provide increased cushioning to the human body by the absorption of energy, is able to provide increased compression to the human body, or both, compared to the article in an unstressed state.

### BRIEF DESCRIPTION OF THE DRAWINGS

The present application may be more completely understood in consideration of the following detailed description of various embodiments of the disclosure in connection with the accompanying drawings.

FIG. 1 is a cross-sectional view of an embodiment of an article of this disclosure,

FIG. 2 is a cross-sectional view of an embodiment of another article of this disclosure.

FIG. 3 is a top view of the article of FIG. 1 prior to lamination.

FIG. 4 is a top view of the article of FIG. 2 prior to lamination.

FIG. 5 is cross-sectional view of an embodiment of another article of this disclosure.

FIGS. 6A and 6B are top views of arrays of cuts in embodiments of articles of this disclosure.

FIG. 7 is a cutting pattern used in Examples 1 and 2.

FIG. 8 is a cutting pattern used in Example 4.

In the following description of the illustrated embodiments, reference is made to the accompanying drawings, in which is shown by way of illustration, various embodiments in which the disclosure may be practiced. It is to be understood that the embodiments may be utilized and structural changes may be made without departing from the scope of the present disclosure. The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

A wide range of applications for cushioning articles have been developed. One area of particular interest for cushioning articles is the medical field, where the need to provide cushioning for joints, limbs, wounds, pressure points and the like are necessary. In some examples, it is also desirable that the cushioning articles also provide compression properties. Examples of compression articles compression stockings, compression sleeves, compression wraps, compression garments, compression tapes, compression bandages, compression dressings, and the like.

Medical articles that provide cushioning are used in first aid circumstances to protect injuries from further injury as well as for therapy and other long term uses. Examples of needs for cushioning articles include pressure injuries. A pressure injury is localized damage to the skin and/or underlying soft tissue, usually over a bony prominence or related to a medical or other device. The injury can present as intact skin or an open ulcer and may be painful. The injury occurs as a result of intense and/or prolonged pressure or pressure in combination with shear.

Frequently cushioning articles use pockets of enclosed air to provide the cushioning and thus are bulky, and relatively inflexible. It therefore is difficult to wrap such articles around joints and injuries. Additionally, the bulkiness of articles requires large storage space. In some instances, cushioning articles can contain deflated pockets that can be inflated when used, but these articles require complex manipulations by the user, and once inflated, the article again is relatively inflexible and difficult to wrap around joints and injuries.

Cushioning is used to provide protection to injured areas of the body such as cuts, scrapes, contusions, bruises and the like to prevent additional injury. Cushioning is also used to protect joints from impact with the ground such as heels, knees, and the like. Pressure-relieving devices such as cushioning articles are particularly suitable for instances where patients are placed on a support surface.

Medical articles that provide compression are used in a wide variety of applications. Compression articles are used to apply pressure to a specific area or injury. They help minimize swelling by keeping fluids from gathering at the injury site. Compression can also be applied through the use of compression stockings or compression sleeves, typically used for long term pain or blood circulation management. Among the common conditions where compression wrapping is used include: wrist or ankle sprains: muscle strains; swollen limbs; varicose veins; and contusions or bruises.

Therefore, it is desirable to have sheet-like articles that can be expanded upon demand to form cushioning articles as needed and are sufficiently flexible to be able to be wrapped around a joint or the injured area of human body. Wrapping with a cushioning article can be used to protect against injury, cushion against long term contact, and in some instances provide compression.

Disclosed herein are sheet-like cushioning articles. In some embodiments, the articles comprise a sheet-like conformable layer that is expandable, comprising a length in an x direction, a width in a y direction and thickness in a z direction. The layer comprises a plurality of cuts, where the plurality of cuts is arrayed in a pattern, and the cuts are gaps formed without the removal of material from the layer and the gaps do not form apertures allowing one to see through the layer when the layer is in an unstressed state, but in a stressed state, when stress is applied in either the x or y direction, the article becomes an expanded article where at least some of the cuts become gaps that are perforations through the layer such that one can view through the layer. The layer expands in the z direction such that the article, when disposed on a human body, is able to provide increased cushioning to the human body by the absorption of energy and increased breathability as measured by MVTR (Moisture Vapor Transmission Rate), compared to an article that is in an unstressed state.

In other embodiments, the articles comprise a multi-layer article with a first layer and a second layer in contact with the first layer, where the second layer is thicker than the first layer, and where the second layer comprises a plurality of apertures, where the apertures are prepared by forming an array of cuts in the second layer without removing material from the second layer and applying stress to the second layer causing the cuts to form apertures. The first layer holds the second layer in a stressed state such that the article, when disposed on a human body, is able to provide increased cushioning to the human body by the absorption of energy, is able to provide increased compression to the human body, or both, compared to the article in an unstressed state.

Thus, there are in general three different scenarios disclosed herein for forming cushioning articles from relatively flat ones by applying stress to the articles. In the first scenario, the cuts formed in a layer form segments that expand largely in the x-y plane. In the second scenario, the cuts form segments that rotate out of the x-y plane. These segments rotate in the z direction and form an angle with the x-y plane of greater than 0° but less than 90°. In the third scenario, the cuts form segments that rotate orthogonally out of the x-y plane, meaning that the segments are rotated in the z direction to form an angle of roughly 90° with the x-y plane.

As used herein, the term “sheet-like” is used according to the commonly understood definition of the term and refers to a three-dimensional article having a length (or x dimension) a width (or y dimension) and a thickness (or z direction) where the x and y dimensions are much larger than the z dimension.

The term “cut” as used herein refers to a narrow opening made in a substrate by the penetration of a cutting tool such that no material is removed from the substrate by the process of cutting. The terms “cuts” and “slits” are used interchangeably.

The term “pattern” as used herein refers to a plurality of cuts, where the plurality of cuts forms an array, and the array is in a pattern, where the pattern may be a “random pattern”, meaning that there is no readily discernible repeating unit in the array, or the array may be aligned along at least one axis. In some embodiments, the array is aligned along one axis, in other embodiments, the array is aligned along more than one axis. The pattern of slits may be single slits, multi-slits, compound slits, orthogonal slits, or a combination thereof.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

The term “adjacent” as used herein when referring to two layers means that the two layers are in proximity with one another with no intervening open space between them. They may be in direct contact with one another (e.g. laminated together) or there may be intervening layers.

The term “gap” as used herein refers to a void space in a substrate that passes through the entire thickness of the substrate. Gaps can be prepared by cutting, slitting, etc. In the current disclosure, one is not able to see through the gaps when the substrate is in an unstressed state, and in a stressed state one is able to see through the gaps as the gaps form “apertures” when in a stressed state. The term “aperture” as used herein is used according to the typical definition and is a space through which light passes. The term “hole” as used herein refers to a void space in the surface of a substrate that is visible to the naked eye in an unstressed state and a stressed state, being an aperture under both an unstressed state and a stressed state.

The term “flap” as used herein refers to a segment of a substrate that upon application of stress is able to move out the x-y plane of the article.

The term “wrapping” as used herein refers to surrounding a portion of a human body and may or may not include overlapping of layers of the article.

The term “tension activation” and “activation upon demand” are used interchangeably herein and refer to applying tension to a layer by wrapping, bending, stretching, pulling or a combination thereof, to form a cushioning article with gaps.

The term “adhesive” as used herein refers to polymeric compositions useful to adhere together two adherends. Examples of adhesives are pressure sensitive adhesives and gel adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

As used herein, the term “gel adhesive” refers to a tacky semi-solid crosslinked matrix containing a liquid or a fluid that is capable of adhering to one or more substrates. The gel adhesives may have some properties in common with pressure sensitive adhesives, but they are not pressure sensitive adhesives. “Hydrogel adhesives” are gel adhesives that have water as the fluid contained within the crosslinked matrix.

As used herein, the term “self-adhesive” refers to a material that adheres to itself but does not appreciably adhere to other substrates. The self-adhesive material surface is non-tacky but when contacted to another layer of the self-adhesive material it has an acceptable adhesive strength. The self-adhesive material adheres by affinity.

Disclosed herein are cushioning articles. In some embodiments, the articles are sheet-like articles that are expandable upon demand by tension activation. In other embodiments, the articles are multi-layer articles with an expanded layer and at least one additional layer that maintains the expanded layer in the expanded condition. These articles are described in greater detail below.

Disclosed herein are cushioning articles that are suitable for a wide range of medical applications. In some embodiments, the cushioning articles comprise a sheet-like conformable layer that is expandable, comprising a length in an x direction, a width in a y direction and thickness in a z direction. The layer comprises a plurality of cuts, where the plurality of cuts is arrayed in a pattern, and where the cuts are gaps, the gaps are formed without the removal of material from the layer and the gaps do not form apertures allowing one to see through the layer when the layer is in an unstressed state, but in a stressed state, when stress is applied in either the x or y direction, the article becomes an expanded article. In the expanded article, at least some of the cuts become gaps that are perforations through the layer such that in a stressed state the perforations are apertures through which one can view through the layer. The layer expands in the z direction such that the article, when disposed on a human body, is able to provide increased cushioning to the human body by the absorption of energy and increased breathability as measured by MVTR (Moisture Vapor Transmission Rate), compared to an article that is in an unstressed state.

The sheet-like conformable layer that is expandable can have a wide range of shapes and sizes depending upon the desired use for the article. Because the sheet-like articles are relatively thin (z direction), they can be conveniently stored and transported. The layer may be prepared from a wide range of materials. In some embodiments, the sheet-like layer comprises a foam, a woven, knitted or a nonwoven web, a rubber, or an energy absorbing material and may include elastomeric filaments arrayed along the x direction. The sheet-like layer may be coated or impregnated with a binder or otherwise comprise a binder. The binder may be a polymeric binder. The binder may be adapted such that a portion of its surface adheres to another portion of its surface when the portions are brought into contact. This property is referred to as “self-adhesive”. The polymeric binder may be an elastomeric polymeric binder or a non-elastomeric polymeric binder. Suitable elastomeric polymeric binders may comprise natural rubber latex, a synthetic latex, such as homopolymer and copolymer latexes of acrylics, butadienes, styrene/butadiene rubbers, chloroprenes, ethylenes (e.g., vinyl acetate/ethylene), isoprenes, nitriles and urethanes, or mixtures thereof. Examples of suitable polymeric elastomeric binders are disclosed for example in U.S. Pat. Nos. 3,575,782; 4,984,585; and 6,155,424.

The sheet-like layer comprises a plurality of cuts, where the plurality of cuts is arrayed in a pattern. In some embodiments, the pattern may be random, in other embodiments the pattern may be arrayed along at least one axis. The cuts are not perforations because they are not formed by removing material from the substrate layer, but rather are cuts are gaps as described above.

The use of cuts rather than perforations has a variety of advantages. Among the advantages is that the cuts are able to reduce the effective modulus without removing material from the sheet-like layer. The array of cuts also form the layer into a cushioning article upon the application of stress to the layer as described below. The array of cuts also provides improved MVTR.

The plurality of cuts can be made in a number of different ways as long as the method does not involve removing material from the sheet-like layer and the cuts form gaps. Among the methods are those in which the cuts are introduced into the sheet-like layer when the layer is formed for example by extrusion, molding, machining and the like. Other methods are those in which the cuts are introduced into the layer after the layer is formed such as by cutting using a cutting tool such as a knife, a linear blade, a rotary die blade, a water jet, or a laser beam, or by stamping using a stamping tool. In some embodiments, the cuts are made by feeding the sheet-like layer into a nip containing a rotary die blade and an anvil such that the die cuts through the sheet-like layer to form the cut pattern.

In some embodiments, the plurality of cuts is arrayed in a pattern along one axis or more than one axis. The plurality of cuts comprises patterns of single slits, multi-slits, compound slits, orthogonal slits, and combinations thereof. The plurality of cuts, upon the application of stress form shaped gaps that indicate the magnitude of the stress applied to the article.

A wide variety of methods can be used to form the plurality of cuts. Among the suitable methods are extrusion, molding, laser cutting, water jetting, machining, stereolithography or other 3D printing techniques, laser ablation, photolithography, chemical etching, rotary die cutting, stamping, other suitable negative or positive processing techniques, or combinations thereof.

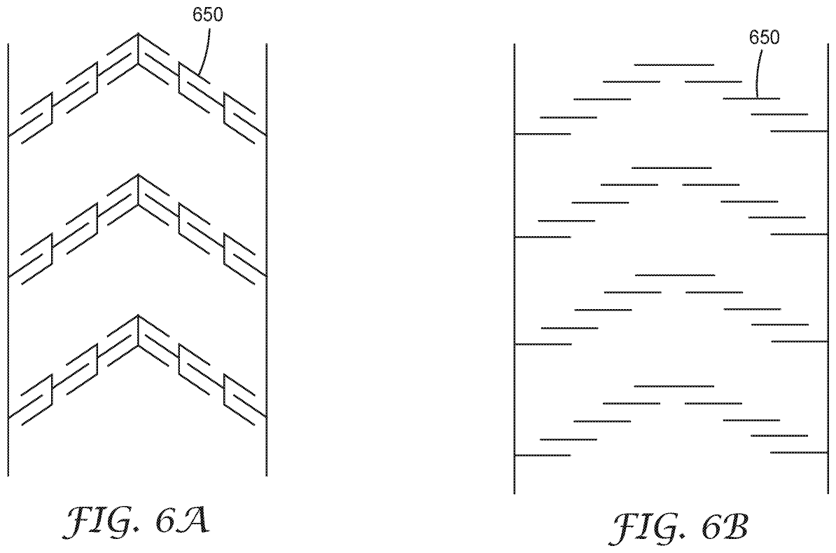

In some embodiments, the cuts are arrayed in a pattern such that the uncut region of the article defines a shape, where the shape is a chevron. In these patterns, the cuts can be single slits, multi-slits, compound slits, orthogonal slits, or combinations thereof. Examples of these shapes are described below and are shown in FIGS. 6A and 6B.

Various embodiments of the present disclosure relate to multi-slit patterns and to articles including these multi-slit patterns.

As used herein, the term “double slit pattern” refers to a pattern of a plurality of individual slits. Each slit in the plurality can be formed by a single continuous cut that does not crossover or intersect itself. The pattern includes a plurality of rows of slits and the individual slits in a first row are substantially aligned with the individual slits in a directly adjacent, second row. A double slit is comprised of a slit in a first row that is substantially aligned with a slit in a second row. Together, these two substantially aligned slits form a double slit.

As used herein, the term “triple slit pattern” refers to refers to a pattern of a plurality of individual slits. Each slit in the plurality can be formed by a single continuous cut that does not crossover or intersect itself. The pattern includes a plurality of rows of slits and the individual slits in a first row are substantially aligned with the individual slits in a directly adjacent, second row. The slits in the second row are substantially aligned with the individual slits in a directly adjacent, third row. A triple slit is comprised of a slit in a first row that is substantially aligned with a slit in a second row, both of which are substantially aligned with a slit in a third row. Together, these three substantially aligned slits form a triple slit.

As used herein, the term “quadruple slit pattern” refers to a pattern of a plurality of individual slits. Each slit in the plurality can be formed by a single continuous cut that does not crossover or intersect itself. The pattern includes a plurality of rows of slits and the individual slits in a first row are substantially aligned with the individual slits in a directly adjacent, second row. The slits in the second row are substantially aligned with the individual slits in a directly adjacent, third row. The slits in the third row are substantially aligned with the individual slits in a directly adjacent, fourth row. A quadruple slit is comprised of a slit in a first row that is substantially aligned with a slit in a second row, both of which are substantially aligned with a slit in a third row, all three of which are substantially aligned with a slit in a fourth row. Together, these four substantially aligned slits form a quadruple slit.

The term “multi-slit pattern” includes double slit patterns, triple slit patterns, quadruple slit patterns, etc. Further, the term “multi-slit pattern” is meant to include any slit pattern wherein two or more slits that are each in different, directly adjacent rows substantially align with one another such that their terminal ends substantially align. Substantial alignment of the terminal ends of aligned multi-slits means that if you draw an imaginary line between two aligned terminal ends in two adjacent slits of the multi-slit, the angle of that imaginary line relative to the alignment axis (the axis that is perpendicular to the row(s)) is no greater than +/−20 degrees. In some embodiments, the length of each slit that forms a multi-slit differs by no more than +/−20% of the total length of the longest or shortest slit. In some embodiments, where the slits are linear, they are substantially parallel to one another. In some embodiments where the slits are not linear, the aligned multi-slits are all substantially aligned parallel to the tension axis within +/−20 degrees.

Double, triple, quadruple, or multi-slit patterns create significantly more out of plane undulation than single slit patterns when exposed to tension along a tension axis. This out of plane undulation of the material has great value for many applications. For example, these out of plane undulation areas create out of plane material or loops that can interlock with other areas of out of plane material or loops when portions of the material are placed adjacent to one another or wrapped together. The undulations also provide useful flat regions, parallel to the original x-y plane, for bonding to adjacent layers of material. As such, multi-slit patterns inherently interlock and/or include interlocking features. Once tension-activated, these features and patterns interlock and hold the material substantially in place. Examples of the wide range of suitable patterns are demonstrated in a series of patterns filed on the same day (Dec. 23, 2019) with the Attorney Docket Numbers: 82095US002; 82539US002; 82540US002; 82541US002; 82542US002; and 82543US002.

Upon application of stress to the sheet-like layer, the material expands. The application of stress comprises wrapping, bending, stretching, pulling or a combination thereof. The application of stress causes one or more of (1) the cuts to form openings and/or (2) the material adjacent to the cuts to rotate out of the plane of the sheet-like article to form segments of the article that can be viewed as flaps. In these embodiments, applying stress to the article causes the material to change from a two-dimensional structure to a three-dimensional structure. In some embodiments, when exposed to stress, the stress causes rotation in the segments of the sheet-like layer between cuts, this rotation occurring out of the x-y plane of the sheet-like article. In some embodiments, these out of plane segments are referred to as flaps and these flaps have a flap shape that is at least one of scale-shaped, curved, rectangular, pointed, cusp-shaped, or combinations thereof.

In some embodiments, if the application of stress is continued, the out of plane rotation can cause the segments to be essentially orthogonal to the x-y plane of the sheet-like article. By essentially orthogonal it is meant that the angle formed by the rotated segment and the x-y plane is close to 90°. The angle may be 90° or within 10 degrees of 90°, or even within 5 degrees of 90°.

In some embodiments, if the application of stress is continued, the segments that rotate out of plane rotate to be essentially orthogonal to the x-y plane of the sheet-like article comprise a large portion of the surface area of the original sheet. Because the portions that rotate orthogonal to the original plane are the most useful portions for absorbing energy and providing cushioning, it is advantageous to maximize the portion of the sheet that rotates orthogonal to the original x-y plane. The portion of the original sheet that becomes orthogonal to the original x-y plane may be greater than 50% or even greater than 80%. In some cases, the portion of the original sheet that becomes orthogonal to the original x-y plane sheet are greater than 90%.

As mentioned above, there are essentially three scenarios disclosed to form cushioning articles. In the first scenario, the application of stress to the article causes the segments formed by cuts in the article to expand in the x-y plane. In the second scenario, the segments formed by cuts in the article expand out of the x-y plane. In the third scenario, the segments formed by curs in the article expand orthogonally out of the x-y plane of the article.

In some embodiments, the sheet-like layer stays in the original x-y plane when expanded and in some embodiments the sheet-like layer rotates out of the original x-y plane when expanded. In general, sheets that are more elastic and thicker will tend to stay in the original x-y plane. The unique patterns shown that enable rotation orthogonal to the original x-y plane automatically deploy into the orthogonal state when tension is applied most effectively when they are made of less elastic materials and thinner. Additional forces can also be applied to a sample to encourage it to either rotate or not rotate depending on the application.

In some embodiments, the sheet-like layer comprises the entire article and can be used to form the cushioning article. In these embodiments. The sheet-like layer is expanded and contacted to a human body to provide cushioning to a portion of the human body. In some embodiments, the article is expanded by stretching and then contacted to the human body. In other embodiments, the sheet-like layer is wrapped around a portion of the human body and the wrapping provides stress activation.

In some articles, the article may comprise one or more additional layers besides the sheet-like layer. In some embodiments, an adhesive layer is present on at least one of the surfaces of the x-y plane. Suitable adhesive layers include pressure sensitive adhesive layers and gel adhesive layers. The adhesive layer can be used to attach the cushioning article to the human body. The adhesive layer can also be used to help attach layers of the cushioning article to each when the article is wrapped.

In some articles, the sheet-like layer is prepared from a self-adhesive material. The self-adhesive material can permit the article to adhere to itself, to help attach layers of the cushioning article to each when the article is wrapped. This self-adhesive effect is utilized in some medical articles, such as some elastic bandages, that upon wrapping the layers self-adhere.

In some embodiments, the stressed state for the article is maintained by attachment of the article to a human body, either through adhesive attachment to the surface of the human body, or by self-adhesion of layers of the article by, for example, wrapping the article around a portion of the human body. In these embodiments, the stressed state is maintained by interaction of mechanical interlocking between layers of the article.

In other embodiments, the stressed state can be maintained without the need for attachment to a human body. This is particularly true of articles where the out of plane rotation can cause the segments to be essentially orthogonal to the x-y plane of the sheet-like article. In these embodiments, the orthogonal segments tend to stay in the stressed state even upon release of applied stress.

Also disclosed herein are multi-layer cushioning articles. Typically, these multi-layer articles are medical articles. In some embodiments, the multi-layer medical articles comprise a first layer and a second layer in contact with the first layer, where the second layer is thicker than the first layer, and where the second layer comprises a plurality of apertures, where the apertures are prepared by forming an array of cuts in the second layer without removing material from the second layer and applying stress to the second layer causing the cuts to form apertures. The first layer holds the second layer in a stressed state such that the article, when disposed on a human body, is able to provide increased cushioning to the human body by the absorption of energy, is able to provide increased compression to the human body, or both, compared to the article in an unstressed state.

A wide range of materials are suitable for use as the second layer. Examples of suitable materials include the materials described above for the sheet-like layer. Particularly suitable are foam materials since foam materials have some cushioning properties even in an unstressed state. The second layer is a relatively thick layer having a thickness of greater than 1,000 micrometers.

Similarly, a wide range of materials are suitable for use in the first layer. In some embodiments, the first layer is a non-woven or a textile layer. In other embodiments, the first layer comprises a film layer prepared from polymeric materials. Representative examples of polymeric materials include polyesters (e.g., polyethylene terephthalates and polyethylene naphthalates), polycarbonates, poly(meth)acrylates (e.g., polymethyl methacrylates), polyurethanes, polyvinyl alcohols, polyolefins such as polyethylenes and polypropylenes, polyvinyl chlorides, polyimides, cellulose triacetates, acrylonitrile-butadiene-styrene copolymers, and the like. The first layer is thinner than the second layer, typically having a thickness of 25-1,000 micrometers, more typically 25-500 micrometers, 25-250 micrometers, or even 25-50 micrometers.

In some embodiments, the medical articles further comprise a third layer in contact with the second layer. In these embodiments, the third layer, like the first layer, is thinner than the second layer. The materials and thicknesses described above for the first layer are also suitable for the third layer. Like the first layer, the third layer can be a non-woven, a textile, or a film. The first and third layers may be the same or different.

Cuts in the second layer may be in patterns as described above for the sheet-like layers, and the techniques to form the cuts described above likewise are suitable.

Upon application of stress to the second layer, the material expands. The application of stress comprises wrapping, bending, stretching, pulling or a combination thereof. The application of stress causes one or more of (1) the cuts to form larger openings and/or (2) the material adjacent to the cuts to rotate out of the plane of the sheet-like article to form segments of the article that can be viewed as flaps. In these embodiments, applying stress to the article causes the material to change from a two-dimensional structure to a three-dimensional structure. In some embodiments, when exposed to stress, the stress causes rotation of the segments of the second layer between cuts, this rotation occurring out of the x-y plane of the second layer. In some embodiments, these out of plane segments are referred to as flaps and these flaps have a flap shape that is at least one of scale-shaped, curved, rectangular, pointed, cusp-shaped, or combinations thereof.

In some embodiments, the application of stress to the second layer causes the thickness of the second layer to decrease in at least some regions of the second layer. In other embodiments, the application of stress to the second layer causes the thickness of the second layer to increase in at least some regions of the second layer. Generally, at least a portion of the increase in thickness is caused by the rotation of portions of the second layer out of the original plane of the un-stressed second layer. In some embodiments, if the application of stress is continued, the out of plane rotation can cause the segments to be essentially orthogonal to the x-y plane of the second layer. By essentially orthogonal it is meant that the angle formed by the rotated segment and the x-y plane is close to 90°. The angle may be 90° or within 10 degrees of 90°. In other embodiments, the application of stress to the second layer causes rotation within the original plane of the un-stressed second layer of at least a portion of the array of cuts essentially without deformation, necking or thickness changes of the rotating elements at elongations to at least 130% of the original layer length. In some embodiments, the elongation is at least 150%.

The stressed second layer is then contacted to the first layer, such that the first layer is able to hold the stressed second layer in the stressed state. In some embodiments, the stressed state is maintained by permanently bonding the first layer to the second layer. In some embodiments, the first layer can be laminated to the second layer. This lamination can be achieved in a variety of ways, including use of an adhesive or by extruding the first layer on the second layer. When an adhesive is used, the adhesive can be disposed either on portions of the first layer or of the second layer. Lamination of the layers can be used to adhere the first layer to the second layer. Alternatively, the first layer can be extruded onto the second layer. In other embodiments, the stressed state is changeable and held temporarily by a non-permanent attachment between the first layer to the second layer in its stressed state.

The medical articles can be used to provide cushioning, compression or a combination thereof to a portion of the human body. In some embodiments, the medical article is disposed on the human body by wrapping with or without overlapping layers. In other embodiments, the medical article can be disposed on the human body by adhesive attachment.

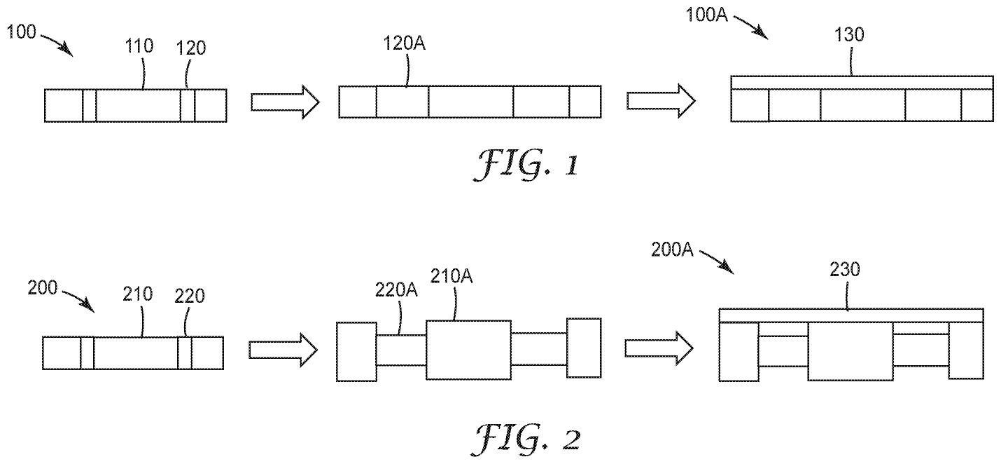

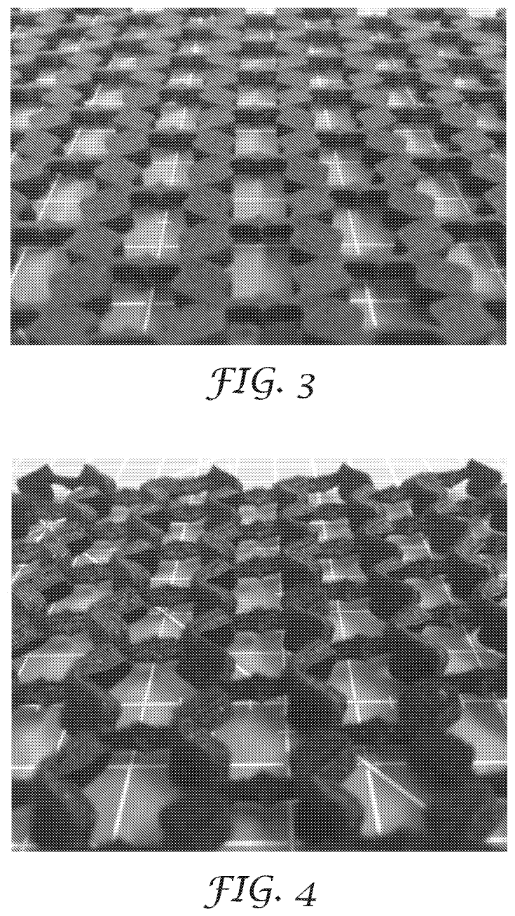

The articles of the current disclosure can be more fully understood by reference to the figures. FIG. 1 is a cross-sectional view of a multi-layer embodiment of an article of this disclosure. FIG. 1 shows article 100 with segments 110 and connection elements 120 between segments 110, the connection elements upon the application of stress become gaps. Generally, the article 100 is a foam article. Stress is applied to article 100 to form article 100A with gaps 120A between segments 110A. While article 100A is maintained in the stressed state, layer 130 is contacted to it to maintain layer 100A in a stressed state. FIG. 2 is a cross-sectional view of an embodiment of another embodiment of an article of this disclosure. FIG. 2 shows article 200 with segments 210 and connection elements 220 between segments 210, the connection elements upon the application of stress become gaps. Generally, the article 200 is a foam article. Stress is applied to article 200 to form article 200A with gaps 220A between segments 210A, where segments 210A are rotated out the plane of article 200A. While article 200A is maintained in the stressed state, layer 230 is contacted to it to maintain layer 200A in a stressed state.

FIG. 3 is a top view of the article of FIG. 1 prior to lamination.

FIG. 4 is a top view of the article of FIG. 2 prior to lamination.

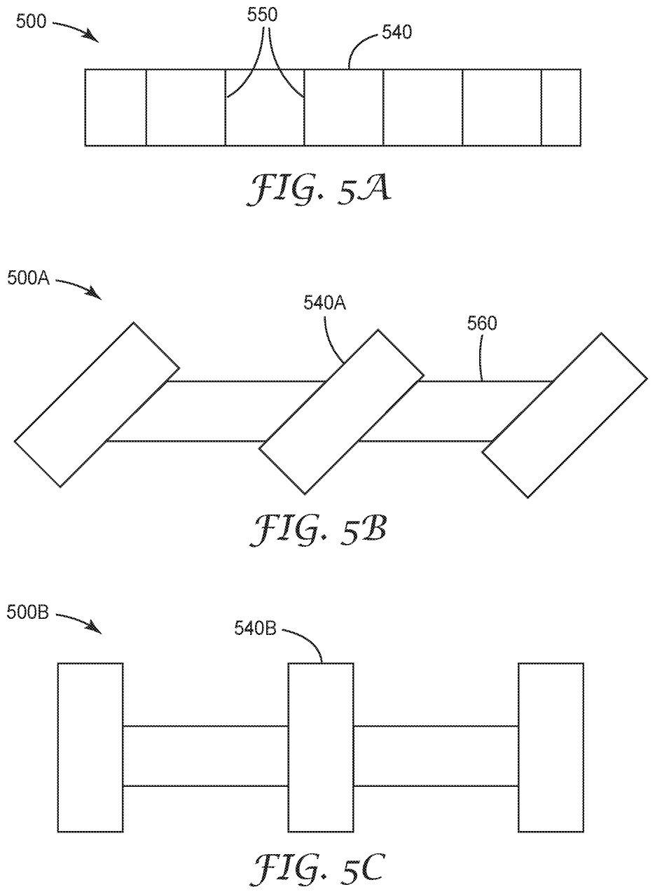

FIG. 5 is cross-sectional view of an embodiment of a sheet-like article of this disclosure. FIG. 5 shows sheet-like article 500 with cuts 550 that define segments 540. Stress is applied to article 500 to form article 500A causing segments 540 to rotate out of the plane of article 500 and form gaps 560. Upon continued application of stress, the article forms segments 540A that are essentially orthogonal to the plane of the article 500 and gaps 560. The stressed article 500 is also exemplified by the article shown in FIG. 4.

FIGS. 6A and 6B are top views of arrays of cuts in embodiments of article of this disclosure. FIG. 6A shows an array of multi-slits, FIG. 6B shows an array of single slits. The space between the patterns of slits are in the shape of a chevron.

### Examples

These examples are merely for illustrative purposes only and are not meant to be limiting on the scope of the appended claims. The following abbreviations are used: cm=centimeters; in-inch; mm=millimeters.

Table of Abbreviations

| Abbreviation | |
|---|---|
| or Trade | |
| Designation | Description |
| Foam-1 | 3 mm New AIRFLEX 2 foam laminate (Eastex Products Inc., |
| | Plymouth, MA, USA), a laminate having the structure Nylon unbroken |
| | loop fabric (UBL)/Polyurethane (PU) foam/Nylon Spandex. The |
| | Nylon UBL was #5283 and comprised 89% Nylon and 11% Spandex. |
| Foam-2 | Foam laminate: VISCO TRI-LAM; GT P-SR-4816 from Rubberlite |
| | Huntington, WV, USA with layers: 1.6 mm thick viscoelastic PU foam |
| | of higher density, (0.063 inch T0812); 2.4 mm thick viscoelastic PU |
| | foam of lower density (0.094″ V0575); and GT P-SR-4816 Silver. The |
| | silver fabric layer was removed manually prior to use. |
| Spray Adhesive | 3M SUPER 77 Spray Adhesive (3M Company, St. Paul, MN) |
| Fabric-1 | WW 983, an open-patterned fabric with hook engaging low profile |
| | loop surface with high stretch resistance (Gehring-Tricot Corp., |
| | Hauppauge, NY, USA) |
| Fabric-2 | A highly densified nonwoven fabric having high stretch resistance |
| Dressing-1 | Tegaderm Transparent Film Dressing, 3M Company, St. Paul, MN, |
| | USA |
| SLL-1 | Sheet-like Layer of neoprene, 1/16 inch (1.59 mm) thick multipurpose |
| | neoprene rubber sheet from McMaster-Carr Supply Company, |
| | Elmhurst, IL, USA. |

### Example 1

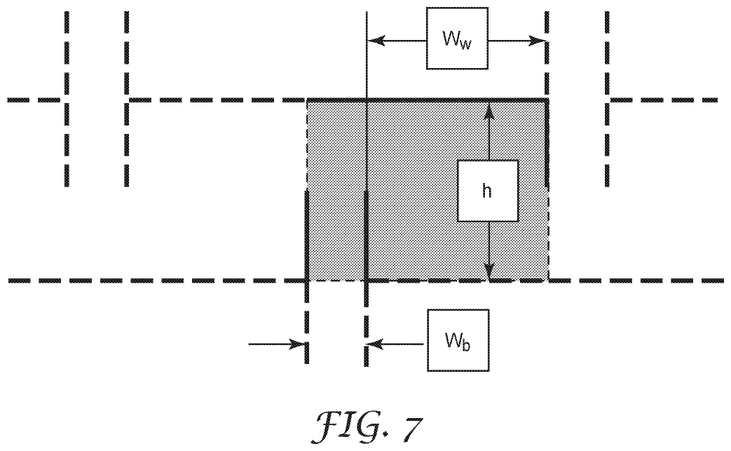

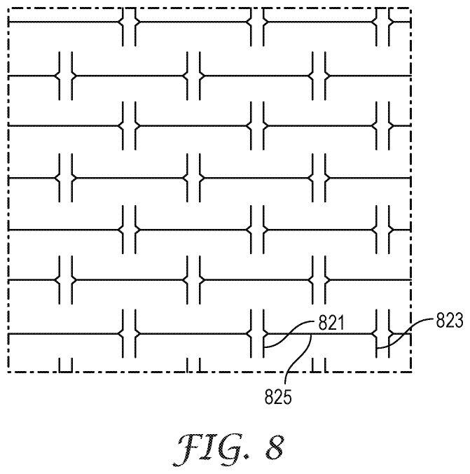

Example 1A used Foam-1 and Example 1B used Foam-2. Foam laminate samples were laser cut using a 60 Watts CO2 Laser, Gravotech Model LS900 (Gravotech, Inc., Duluth, GA, USA). The laser system was operated at 20% speed and 15% power. The foam laminate samples measured 12 cm (y axis)×26 cm (x axis). The cut pattern and parameters are shown in FIG. 7. The pattern was cut with rounded corners with a radius of curvature of 1 mm as shown in FIG. 8.

Cutting parameters:
h=6 mm
Ww=6.5 mm
Wb=1.5 mm

### Results:

### Example 1A

The cut foam of Example 1A (Foam-1) was elongated approximately 100% in the x-axis direction, and the orthogonal pop-up effect was observed. The cut foam was stretched to approximately 200% of initial length and the resultant article is shown in FIG. 4.

### Example 1B

The cut foam of Example 1B (Foam-2) was elongated approximately 100% in the x-axis direction, and very little orthogonal pop-up effect was observed. The cut foam was stretched to approximately 200% of initial length and the resultant article is shown in FIG. 3.

### Example 2: Laminations of a Cut Foam Laminate to Uncut Stretch-Resistant Fabrics

The cut foam from Example 1B above was stretched by approximately 80% (to 180% of initial length) and was kept in this state with the help of weights laid on the ends of the sample. The surface was then sprayed with Spray Adhesive. After two minutes, the second layer (for Example 2A the second layer was Fabric-1; for Example 2B the second layer was Fabric-2) was laid without tension on the prepared surface of the elongated foam laminate and was pressed on manually. After another two minutes, the loads were removed. Upon removal of the weights, the laminate showed only little elastic rebound.

### Example 3

The cut foam from Example 1B was draped around a 3-dimensional surface, i.e., the heel of a leg model, stretching as necessary to conform to the surface of the heel. While maintained in this position, a stretchable adhesive film of Dressing-1 was placed with minimal tension over the laminate and manually adhered. When the completed construction was removed from the heel, it maintained its shape.

### Example 4

A Tension Activated Expanding Sheet (TAES) was prepared by laser cutting into a sheet of SLL-1 of 14 in (36 cm)×201 in (51 cm) using a modification of the pattern shown in FIG. 7, where the modifications are shown in FIG. 8. The parameters for the pattern (as described by FIG. 7) are shown below.

### Parameters:

h=10 mm
Ww=10 mm
Wb=2.5 mm

The pattern of FIG. 8 is a top view schematic drawing of a compound slit pattern that is substantially the same as the compound slit pattern of FIG. 7 except that the intersections between the generally horizontal slit portion 825 and the two generally vertical slit portions 821, 823 are rounded or have rounded corners. These features soften the edges of the material by removing sharp corners that users might encounter during use of the material.

### Ball Drop Test

The resilience and energy absorption capacity of the film was evaluated by dropping a ball and measuring the height of the bounce for N number of trials. The ball was a rubber ball of 52 grams and a diameter of 2.37 in (60 mm) and was dropped from a height of 66 in (1680 mm). The surface tested was a bare concrete floor, the TAES in an undeployed (flat, as cut) state on the concrete floor, and the TAES deployed by extension to 150% of the undeployed state, sufficient to deploy the orthogonal pop-up state. The results of the testing are provided in Table 1, and show a significant increase in the absorption of energy was obtained with the expanded sheet relative to the flat sheet.

TABLE 1
Results of Ball Drop Test.

| | No pad (concrete floor) | Flat Sheet | Deployed Sheet |
|---|---|---|---|
| Average | 41.4 in (105 cm) | 40.9 in (104 cm) | 12.8 in (33 cm) |
| height | (N = 10) | (N = 12) | (N = 10) |

1. A cushioning article comprising:
a sheet-like conformable layer that is expandable, comprising a length in an x direction, a width in a y direction and thickness in a z direction, wherein the layer comprises a plurality of cuts, wherein the plurality of cuts is arrayed in a pattern, and wherein the cuts are gaps, the gaps are formed without the removal of material from the layer and the gaps do not form apertures allowing one to see through the layer when the layer is in an unstressed state, but in a stressed state, when stress is applied in either the x or y direction, the article becomes an expanded article wherein at least some of the cuts become gaps that are perforations through the layer such that in a stressed state the perforations are apertures through which one can view through the layer, and wherein the layer expands in the z direction such that the article, when disposed on a human body, is able to provide increased cushioning to the human body by the absorption of energy and increased breathability as measured by MVTR (Moisture Vapor Transmission Rate), compared to an article that is in an unstressed state.

2. The cushioning article of claim 1, wherein at least a portion of the expansion in the z direction is caused by the rotation of portions of the layer out of the original x-y plane of the article prior to the application of stress.

3. The cushioning article of claim 1, wherein at least a portion of the expansion in the z direction is caused by the rotation of portions of the layer that become essentially fully orthogonal to the original x-y plane of the article prior to the application of stress.

4. The cushioning article of claim 1, wherein the plurality of cuts comprises patterns of single slits, multi-slits, compound slits, orthogonal slits, and combinations thereof.

5. The cushioning article of claim 1, wherein the plurality of cuts comprise a pattern such that an uncut region of the sheet-like layer are in the form of chevron shape.

6. The cushioning article of claim 1, wherein the sheet-like layer comprises a foam, a woven, knitted or a nonwoven web, a rubber, or an energy absorbing material and may include elastomeric filaments arrayed along the x direction or may be coated or impregnated with a polymeric binder.

7. The cushioning article of claim 1, wherein the application of stress comprises wrapping, stretching, pulling or a combination thereof.

8. The cushioning article of claim 1, wherein the article further comprises an adhesive layer or comprises a self-adhesive material on at least one of the surfaces of the x-y plane of the article.

9. The cushioning article of claim 8, wherein the stressed state is maintained by the interaction of the adhesive layer and a human body

10. The cushioning article of claim 8, wherein the article is wrapped, and the stressed state is maintained by the interaction of the adhesive layer with a non-adhesive layer of the article or by the interaction between layers of self-adhesive material.

11. The cushioning article of claim 1, wherein the article is wrapped, and the stressed state is maintained by the interaction of mechanical interlocking between layers of the article.

12. A medical article comprising a multi-layer article comprising:
a first layer; and
a second layer in contact with the first layer;
wherein the second layer is thicker than the first layer, and wherein the second layer comprises a plurality of apertures, where the apertures are prepared by forming an array of cuts in the second layer without removing material from the second layer and applying stress to the second layer causing the cuts to form apertures, wherein the first layer holds the second layer in a stressed state such that the article, when disposed on a human body, is able to provide increased cushioning to the human body by the absorption of energy, is able to provide increased compression to the human body, or both, compared to the article in an unstressed state.

13. The medical article of claim 12, wherein the article further comprises a third layer in contact with the second layer, wherein the third layer is thinner than the second layer.

14. The medical article of claim 12, wherein the article is disposed on the human body by wrapping with or without overlapping layers.

15. The medical article of claim 12, wherein the application of stress to the second layer causes the thickness of the second layer to decrease in at least some regions of the second layer.

16. The medical article of claim 12, wherein the application of stress to the second layer causes the thickness of the second layer to increase in at least some regions of the second layer.

17. The medical article of claim 16, wherein a portion of the increase in thickness is caused by the rotation of portions of the second layer out of the original plane of the un-stressed second layer.

18. The medical article of claim 16, wherein a portion of the increase in thickness is caused by the rotation of portions of the second layer that become essentially orthogonal to the original plane of the un-stressed second layer.

19. The medical article of claim 12, wherein the application of stress to the second layer causes rotation within the original plane of the un-stressed second layer of at least a portion of the array of cuts essentially without deformation, necking or thickness changes of the rotating elements at elongations to at least 130% of the original layer length.

20. The medical article of claim 12, wherein the stressed state is maintained by permanently bonding the first layer to the second layer in its stressed state.

21. (canceled)
